(19) United States
(12) Patent Application Publication
(10) Pub. No.: US 2018/0228165 A1
(43) Pub. Date: Aug. 16, 2018

(54) PREPARATION ABLE TO PRODUCE BIOPESTICIDE AND/OR REPELLENT FOR CONTROLLING PLANT PATHOGENS

(71) Applicant: Luxembourg Institute Of Science And Technology (LIST), Esch/Alzette (LU)

(72) Inventor: Bruno PRINTZ, Escherange (FR)

(21) Appl. No.: 15/750,999

(22) PCT Filed: Aug. 3, 2016
(86) PCT No.: PCT/EP2016/068511

§ 371 Date: Feb. 7, 2018

(30) Foreign Application Priority Data
Aug. 7, 2015 (LU) 92793

(51) Int. Cl. A01N 65/00; C12N 9/24; C12N 11/14

(52) U.S. Cl. CPC A01N 65/00; C12N 11/14; C12Y 302/01147; C12N 9/2402

(57) ABSTRACT

A preparation able to produce a biopesticide and/or repellent for controlling plant pathogens, comprising at least one nanoporous material and at least one active enzyme, the at least one active enzyme being configured to transform at least one precursor into a biopesticide and/or repellent product. The preparation able to produce a biopesticide and/or repellent for controlling plant pathogens is remarkable in that the at least one active enzyme is immobilized within the at least one nanoporous material.

### CROSS-REFERENCE TO RELATED APPLICATIONS

The present invention is the US national stage under 35 U.S.C. § 371 of International Application No. PCT/EP2016/068511, which was filed on Aug. 3, 2016, and which claims the priority of application LU 92793 filed on Aug. 7, 2015, the content of which (text, drawings and claims) are incorporated here by reference in its entirety.

### FIELD

The invention is directed to a preparation able to produce a biopesticide and/or repellent for controlling plant pathogens, the preparation comprising an enzyme capable to transform a substrate into a biopesticide and/or repellent product which will subsequently control plant pathogens. The invention also concerns the implementation of such preparation within a pest protection apparatus in order to make use of such preparation in larger scale, for example in greenhouses and/or in private or market gardening.

### BACKGROUND

Pest management is currently largely performed with synthetic chemicals, but the development of alternative and eco-friendly methods to control pests are encouraged.

The field of the biocontrol includes various techniques of integrated pest management such as macro-organisms, micro-organisms, pheromones, kairomones and natural substances originating from animals, minerals and plants to control pest and pathogens. In 2014, Sahayaraj K. summarized the current use of the nanotechnologies in the domain of the plant protection. The few data available on this topic brings to the conclusion that only few research all over the world are currently working with this emerging problematic although there is an urgent need of modern approaches of pest management (Sahayaraj K., Adv. Plant Biopest. (chapter 14), 2014, 279-293).

Substances from various plant families have been reported to exert a repelling activity against fungus, bacteria, nematodes or insects and can be successfully used in the treatment/control of pests (Murthy N. B. K., et al., Indian J. Exp. Biol., 1974, 12, 208-209 and Regnault-Roger C., et. al., Biopesticides d'ongine végétale (2ème edition), 2008.) In particular, sulphur containing molecules such as those produced among other by the families Brassicaceae and members of the genus Allium which are enzymatically produced from non-toxic precursors are toxic at low level for a wide range of organisms (Ahuja I., et al., Agron. Sustain. Dev., 2010, 30, 311-348; Auger J., et al., Ecologie, 1994, 93-101; Cutler H. G., et al., Biologically Active Natural Products: Agrochemicals, 1999).

The mixing of biopesticide precursor with active enzymes in liquid form to produce repellent is known to the art and has been proposed for the production of glucosinolate products such as nitrile, thiocyanate or isothiocyanate, and/or mixture (WO 2015/013808 A1). Also known in the art is the use of a two-part pesticide precursor system comprising a glucosinolate concentrate on one side and an active myrosinase complex on the other side both in dry form. When both components are mixed with water, the glucosinolate breakdown products are released to control pests (US 2015/0005172 A1). However, the lifespan of active enzymes in the environment is relatively low, which considerably hinders the reusability of the enzymes. In addition, the substrates are commonly hydrophilic, making them easily leachable and not accessible to the enzymes.

Enhancing the lifespan of enzymes has been successfully performed in the domains of the biocatalysis, biofuels, enzyme-controlled drug delivery and the biosensors by immobilizing the enzymes into silica mesoporous material (Carlsson N., et al., Adv. Colloid Interface Sci., 2013, 205, 339-360; Popat A., et al., Nanoscale, 2011, 3, 2801-2818; Wang Y., et al., Chem. Mater., 2005, 17 953-961). Mesoporous material relates to silica-based material with a pore diameter sufficient to allow the penetration of the enzyme. It can be in the form of SBA-15 (Santa Barbara-15), SBA-16, MCM-41 (Mobil Crystalline Materials-41) or all other type of mesoporous silica sieves/spheres/cages (FSM-16 (Folded Sheet Mesoporous-16), MCM-48, FDU-12 (Fudan University-12), MCF (Mesostructured Cellular Foam), SMS (Sponge Mesoporous Silica), mesoporous carbon, PMOs (Periodic Mesoporous Organosilica), Meso-MOFs (meso-Metal Organic Frameworks) (Zhou Z., et al., Top Catal., 2012, 55, 1081-1100). The pore structure may be folded-sheet, 2D hexagonal channels, cubic, spherical cages, mesocellular foam, sponge-like mesoporous silica, 3D cubic cages. The synthesis of the mesoporous silica is preferentially done in acidic conditions and with the triblock copolymer (Pluronic®P123) as template and tetraethyl orthosilicate (TEOS) as silica source. However, the use of other templates such as among other the triblock copolymer (Pluronic®F127), CTMA (cetyltrimethylammonium) (CₙH₂ₙ₊₁(CH₃)N⁺, n=8-18), CTAB (cetyltrimethylammonium bromide) and other silica source including among other (3-aminopropyl)trimethoxysilane (APTMOS), sodium silicate or tetramethyl orthosilicate (TMOS) may also give interesting properties to the silica mesoporous material (Zhou Z., et al., Top Catal., 2012, 55, 1081-1100).

Enzyme immobilization into the silica matrix can be carried out through various techniques including cross-linking methods, covalent binding, physical adsorption, encapsulation and entrapment (CN 101451133 B). Carboxylethyl or aminopropyl functionalization of the mesoporous silica also appears as a good technique for improving the catalytic activities of the silica-based biocomposite (Lei C., et al., J. Am. Chem. Soc., 2002, 124, 11242-11243). Functionalization of the silica-material can further be used for covalent linking of the enzyme on the derivatized-silica.

The synthesis of hydrophilic gels can result from the gelation of various natural compounds such as agarose, agar-agar, alginate, pectin, starch or gelatine taken alone or in mixes. They allow the diffusion of small hydrophilic molecules and prevent the diffusion of large particles such as silica mesoporous materials that retain blocked in the polymeric network. Microencapsulation of silica in alginate had remarkable advantages of sustained-release of compounds and stability under different pH values, different temperatures, and UV irradiation. The presence of the hydrophilic gel can further increase the stability of the material upon ageing and limit enzyme leaching (Coradin T., et al., Comptes Rendus Chim., 2003, 6, 147-152). Biocomposite incorporation in hydrophilic gels such as alginate showed much better performance due to the more homogeneous distribution of silica particles in the composite material (Xu S. et al., Ind. Eng. Chem. Res., 2006, 45, 511-517).

In the present invention, these parts (mixing of a precursor with active enzymes in liquid form to produce biopesticide and/or repellent product, enhancing the lifespan of the enzymes, use of hydrophilic gels) are merged together in order to produce for the first time active mesoporous materials encapsulated in a hydrophilic gel in order to produce in situ a constant flux of biopesticide and/or repellent product.

On the other hand, patent application published US 2012/0079625 A1 relates to a method for protecting living plants from harmful insects via a sheet-like structure impregnated with an insecticide which is from synthetic origin.

### SUMMARY

The invention has for technical problem to provide a preparation, able to produce a biopesticide and/or repellent for controlling plant pathogens, which is non-toxic and easy to handle. This will confer interesting storage and manipulation properties while keeping the biopesticide and/or repellent properties of such preparation. The preparation will also be adaptable to several types of configuration, from small one such as granulates or fibres to bigger one, such as pest protection apparatus.

The first object of the present invention is a preparation able to produce a biopesticide and/or repellent for controlling plant pathogens, comprising at least one nanoporous material and at least one active enzyme, the at least one active enzyme being configured to transform at least one precursor into a biopesticide and/or repellent product. The preparation able to produce a biopesticide and/or repellent for controlling plant pathogens is remarkable in that the at least one active enzyme is immobilized within the at least one nanoporous material.

In various embodiments, the at least one nanoporous material is a mesoporous material.

In various embodiments, the at least one nanoporous material is embedded within one hydrophilic gel.

In various embodiments, the hydrophilic gel is made of agarose, agar-agar, alginate, pectin, starch and/or gelatine, in various instances made of alginate.

In various embodiments, the at least one active enzyme is one active enzyme selected from the group of glycosidase, lyase and/or lachrymatory-factor synthase, in various instances from the group of glycosidase.

In various embodiments, the at least one active enzyme from the group of glycosidase is thioglucosidase.

In various embodiments, the at least one active enzyme immobilized within the at least one nanoporous material is covalently bounded to the at least one nanoporous material, physically adsorbed to the at least one nanoporous material, encapsulated within the at least one nanoporous material or entrapped within the at least one nanoporous material.

In various embodiments, the active enzyme immobilized within the at least one nanoporous material is cross-linked together with glutaraldehyde or with 1-ethyl-3-(3-dimethylaminopropyl)carbodiimide and N-hydroxysuccinimide.

In various embodiments, the at least one nanoporous material is a silica-based mesoporous material.

In various embodiments, the silica-based mesoporous material comprises tetraethyl orthosilicate and/or tetramethyl orthosilicate and/or methyltrimethoxysilane and/or (3-aminopropyl)trimethoxysilane.

In various embodiments, the preparation has a pH comprised between 4 and 11.

In various embodiments, the preparation further comprises the at least one precursor, at least one mineral and/or at least one cofactor.

In various embodiments, the preparation is adapted to receive the at least one precursor, at least one mineral and/or at least one cofactor.

In various embodiments, the at least one mineral and/or the at least one cofactor is phosphate buffer or ascorbic acid.

In various embodiments, the phosphate buffer is at a concentration comprised between 10 mM and 500 mM, in various instances at a concentration comprised between 50 mM and 250 mM, for example at a concentration comprised between 75 mM and 150 mM, e.g., at a concentration of 100 mM.

In various embodiments, the ascorbic acid is at a concentration comprised between 50 μM and 1500 μM, in various instances at a concentration comprised between 150 μM and 1000 μM, for example at a concentration comprised between 250 μM and 750 μM, e.g., at a concentration of 500 μM.

In various embodiments, the preparation is dehydrated and/or rehydrated.

It is a second object of the present invention to disclose a fibre adapted to control plant pathogens. The fibre is remarkable in that it is coated with at least one preparation able to produce a biopesticide and/or repellent for controlling plant pathogens in accordance with the first object of the present invention.

In various embodiments, the at least one preparation is further coated with a protective layer, the protective layer being in various instances a hydrophobic layer.

In various embodiments, the hydrophobic layer is made of 3-aminopropyl)triethoxysilane, (3-mercaptopropyl)triethoxysilane, succinic anhydride, alkylketene dimer, 3-isopropenyl-α-α-dimethylbenzyl isocyanate, m-phenylene bismaleimide, vinyl trialkoxysilane, 3-metacryloyloxy propyl trimetoxysilane or any other.

In various embodiments, the fibre is a natural fibre, in various instances hemp, flax, nettle, cotton, jute, ramie, sisal or any other, for example hemp or flax.

It is a third object of the present invention to disclose a pest protective apparatus comprising at least one fibrous network and/or at least one fibrous cover. The pest protective apparatus is remarkable in that the at least one fibrous network and/or the at least one fibrous cover is made of fibres adapted to control plant pathogens, in accordance with the second object of the present invention.

In various embodiments, the pest protective apparatus is configured to transform at least one precursor into a biopesticide and/or a repellent product.

In various embodiments, the pest protective apparatus further comprises means for distributing water to the fibrous network and/or fibrous cover, the means in various instances comprising a cartridge configured for being in fluid connection with the at least one fibrous network and/or the at least one fibrous cover and for being fed with water.

In various embodiments, the cartridge comprises at least one precursor to the preparation able to produce a biopesticide and/or repellent for controlling plant pathogens that is provided in the fibres adapted to control plant pathogens.

In various embodiments, the cartridge further comprises at least one mineral and/or at least one cofactor.

In various embodiments, the cartridge is adapted to receive the at least one precursor, at least one mineral and/or at least one cofactor.

In various embodiments, the at least one mineral and/or the at least one cofactor is phosphate buffer or ascorbic acid.

In various embodiments, the phosphate buffer is at a concentration comprised between 10 mM and 500 mM, in various instances at a concentration comprised between 50 mM and 250 mM, for example at a concentration comprised between 75 mM and 150 mM, e.g., at a concentration of 100 mM.

In various embodiments, the ascorbic acid is at a concentration comprised between 50 μM and 1500 μM, in various instances at a concentration comprised between 150 μM and 1000 μM, for example at a concentration comprised between 250 μM and 750 μM, e.g., at a concentration of 500 μM.

It is a fourth object of the present invention to disclose the teachings of a method for controlling plant pathogens with a preparation able to produce a biopesticide and/or repellent for controlling plant pathogens. The method is remarkable in that it comprises the first step of activating at least one precursor by addition of water to the preparation able to produce a biopesticide and/or repellent for controlling plant pathogens in accordance with the first object of the present invention for producing a biopesticide and/or repellent product for controlling plant pathogens and the second step of applying the biopesticide and/or repellent product resulting from the first step to plants, in various instances vegetables and/or fruits, in order to prevent pest attacks. The first step of activating at least one precursor can also be, for example, the catalytic splitting of the at least one precursor.

In various embodiments, the at least one precursor is comprised into the preparation able to produce a biopesticide and/or repellent for controlling plant pathogens in accordance with the first object of the present invention, the preparation in various instances also comprising at least one mineral and/or at least one cofactor.

In various embodiments, the at least one precursor is added to the preparation able to produce a biopesticide and/or repellent for controlling plant pathogens in accordance with the first object of the present invention, the preparation in various instances comprising at least one mineral and/or at least one cofactor.

In various embodiments, the at least one precursor is added with also at least one mineral and/or at least one cofactor to the preparation able to produce a biopesticide and/or repellent for controlling plant pathogens in accordance with the first object of the present invention.

In various embodiments, the preparation able to produce a biopesticide and/or repellent for controlling plant pathogens is on a fibre adapted to control plant pathogen in accordance with the second object of the present invention, the fibre being in various instances comprised into a pest protective apparatus in accordance with the third object of the present invention.

The invention is particularly interesting in that the precursor or the precursors of the biopesticide and/or repellent product which is provided by the preparation is chosen among the inactive and non-toxic forms of the potential organic derivatives. The immobilization of the enzyme into a nanoporous material, in various instances a silica nanoporous material, for example a silica-based mesoporous material, will further enhance the stability of the core of the preparation able to produce a biopesticide and/or repellent for controlling plant pathogens and will further enhance the productivity of the compound with biopesticide and/or repellent properties.

### DRAWINGS

FIG. 1 illustrates a preparation able to produce a biopesticide and/or repellent for controlling plant pathogens in accordance with various embodiments of the invention.

FIG. 2 illustrates fibres adapted to control plant pathogens in accordance with various embodiments of the invention.

FIG. 3 illustrates a pest protective apparatus in accordance with various embodiments of the invention.

### DETAILED DESCRIPTION

The present invention relates to a pest management biocontrol system made of active biocatalysts able to produce natural repellents and/or biopesticides by means of controlled enzymatic reactions.

The present invention offers to the user an easy-to-handle and safe way to control plant pest by producing a constant flux of biopesticide and/or repellent products, being insecticides, fungicides, bactericides and/or nematicides.

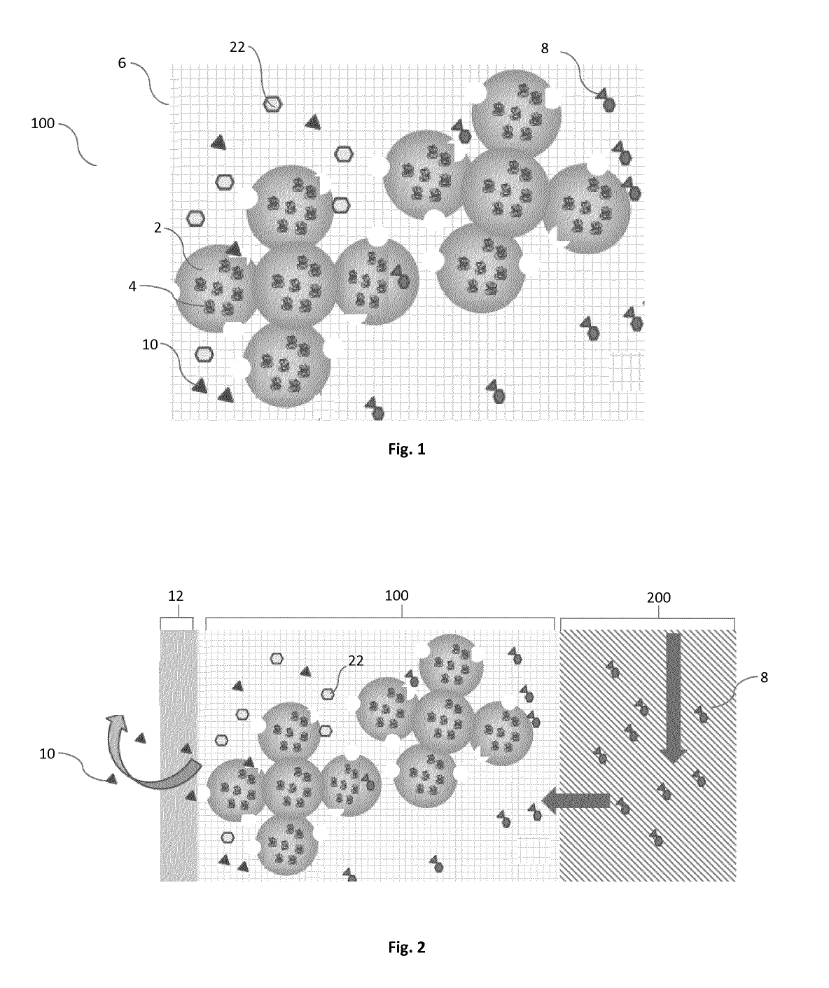

FIG. 1 depicts a preparation 100, able to produce a biopesticide and/or repellent for controlling plant pathogens, according to the present invention.

The biopesticide and/or repellent compounds produced are those involved in the natural reaction of the plants when facing a pathogen attack. They have a low half-life in the environment and do not affect the quality of the plants which are treated.

The present invention relates to an innovative product that entraps enzymes 4 into a nanoporous material 2, in particular a silica sphere, in various instances into a mesoporous silica matrix, more particularly into a silica-based mesoporous material, the whole being glued or embedded into a hydrophilic gel 6, to degrade natural molecules or extracts into products 10 having biopesticide and/or repellent activity.

The mesoporous materials 2 of the present invention are ordered silica-based mesoporous particles with a narrow pore size distribution, a well-defined pore geometry and a well-defined pore connectivity.

The pore size distribution and the global geometry of the mesoporous materials 2 are defined according to the properties of the biopesticide-producing enzymes and/or repellent-producing enzymes that are immobilized into the mesoporous structure.

The pore size of the silica-based mesoporous material is comprised between 5 nm and 30 nm, more particularly 5 and 15 nm for thioglucosidase.

The immobilization procedure should allow to optimize the catalytic activities of the enzymes in comparison with the free enzymes and to enhance their reusability in order to produce a constant and sufficient flux of biopesticide and/or repellent product allowing a sufficient biopesticide and/or repellent activity against the targeted pathogens.

The silica-based mesoporous material will be synthesized in acidic conditions using a silica precursor that could be among other tetraethyl orthosilicate (TEOS) and/or tetramethyl orthosilicate (TMOS) and/or methyltrimethoxysilane (MTMOS) and/or (3-aminopropyl)triemthoxysilane (APTMOS), in combination with tri-block copolymer mixtures such as Pluronic®F127 and/or Pluronic®F123 as structure-directing agents. Numerous methods are known by the skilled person in the art to achieve this synthesis.

The mesoporous structure can be produced hydrothermally and/or by sol-gel synthesis and can be functionalized.

The enzymes 4 that can be immobilized into the mesoporous material are those involved in the natural response of the plants when facing a pathogen attack. The biopesticide and/or repellent compounds produced during the enzymatic reaction can be a bactericide, a fungicide, an insecticide and/or a nematicide.

The enzyme from the glycosidase, the oxidoreductases, the transferases, the hydrolases, the lyases, the isomerases and the ligases can be immobilized into the mesoporous material.

Exemplary transferases are those belonging to the glycosidases class, i.e. the enzymes hydrolysing O-glycosyl and S-glycosyl compounds.

Exemplary lyases are, for example, carbon-sulfur lyases, in particular alliin lyase (also known as alliinase).

Enzymes from the PF10604 family, i.e. the lachrymatory-factor synthase, can also be used in the present invention.

An exemplary glycosidase is thioglucosidase. This is the enzyme of choice which has been tested for transforming a precursor into a biopesticide and/or repellent product.

The immobilization procedure can be performed through the following techniques known in the art: cross-linking methods, covalent binding, physical adsorption, encapsulation and entrapment.

The immobilization includes the penetration of the enzymes inside the mesoporous material, through the nanopores and an eventual step of cross-linkage between the enzymes using gluturaldehyde or 1-ethyl-3-(3-dimethylaminopropyl)carbodiimide and N-hydroxysuccinimide. Numerous methods are known by the skilled person in the art to achieve the immobilization step.

The mesoporous biocatalyst will then be embedded in a hydrophilic gel 6 made of agarose, agar-agar, alginate, pectin, starch, gelatine taken alone or in mixes, in various instances alginate.

The use of alginate allows a gelation without heating. Only CaCl₂ must be added for the gelation step.

The hydrophilic gel 6 allows the diffusion of the products of the enzymatic reaction, the diffusion of the substrates 8 of the enzymes 4 and the confinement of all chemicals required to maintain the enzymes 4 in a working status.

The hydrophilic gel 6 can contain all minerals and cofactors indispensable for the enzymatic activities. This can be a phosphate butter (pH=6.1) at a concentration comprised between 10 mM and 500 mM, in various instances at a concentration of 100 mM and/or ascorbic acid at a concentration comprised between 50 μM and 1500 μM, in various instances at a concentration of 500 μM.

In case the minerals and cofactors are not added before the gelation step, they can be added by diffusion within the gel by soaking into an aqueous buffer comprising these compounds.

The pH of the preparation 100 able to produce biopesticide and/or repellent for controlling plant pathogens is in various instances comprised between 4 and 11. In particular, the pH of the hydrophilic gel allows an optimal activity of the immobilized enzymes.

The hydrophilic gel with the silica-based mesoporous material embedded within the hydrophilic gel, the silica-based mesoporous comprising immobilized thioglucosidase, forms a biopesticide and/or repellent preparation 100 adapted to control plant pathogen.

Upon addition of the substrate 8 for the enzyme 4 and upon addition of water, and, upon addition of minerals and cofactors for activating the enzyme 4 if they are not formerly included within the hydrophilic gel 6, the enzyme will proceed to the synthesis of a product 10 which will present biopesticide and/or repellent properties. This product 10 will be released and will act to eliminate (in case of biopesticide activity) and/or to keep away (in case of repellent activity) the pest and/or the plant pathogens from the living plants, such as bacteria, fungi, insects, bugs, and/or nematodes.

The scheme on FIG. 1 represents in particular a precursor 8 composed of two chemical moieties, one being the biopesticide and/or the repellent product 10, and the other being a compound 22.

In various instances, the product 10 of the enzymatic reaction is a volatile organic compound.

The biopesticide and/or repellent preparation 100 can further be coated onto fibres 200 or natural fibres. This is schematically depicted on FIG. 2.

Such fibres might be for example hemp, flax, nettle, cotton, jute, ramie, sisal, and/or any other.

In various instances, the fibres are hemp or flax.

The substrate 8 of the enzyme 4 is able to diffuse within the fibre 200. This will allow the substrate 8 to reach the preparation 100 able to produce biopesticide and/or repellent for controlling plant pathogens in order to be processed by the enzyme 4.

In order to protect such fibres 200 from drying, a protective layer, in various instances a hydrophobic layer 12 will be coated over the fibres 200 containing a coating of the preparation 100 able to produce biopesticide and/or repellent for controlling plant pathogens.

Such hydrophobic layer 12 is also configured to be permeable to the biopesticide and/or repellent product 10.

The hydrophobic layer 12 can be (3-aminopropyl)triethoxysilane, (3-mercaptopropyl)triethoxysilane, succinic anhydride, alkylketene dimer, 3-isopropenyl-α-α-dimethylbenzyl isocyanate, m-phenylene bismaleimide, vinyl trialkoxysilane, 3-metacryloyloxy propyl trimetoxysilane and/or any other.

The moiety 22, resulting from the cleavage of the precursor 8, stays therefore or diffuses slowly within the preparation 100 able to produce biopesticide and/or repellent for controlling plant pathogens and does not therefore pollute the environment.

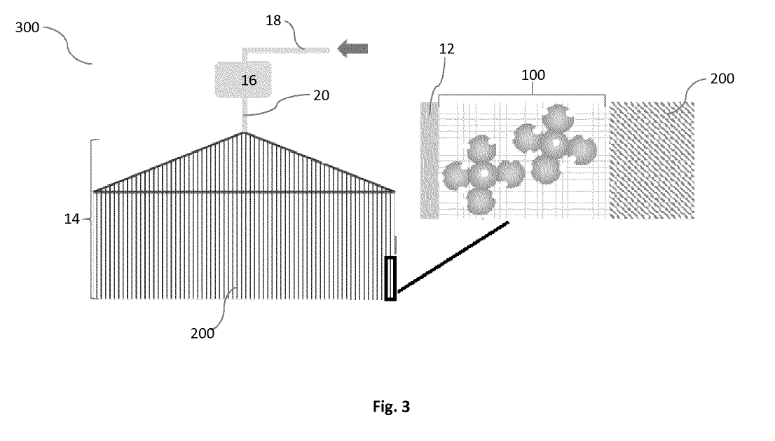

As indicated on FIG. 3, the fibres 200 with the biopesticide and/or repellent preparation 100 might also be incorporated within a pest protective apparatus 300, more particularly by coating onto at least one fibrous network and/or at least one fibrous cover 14 of the pest protective apparatus 300.

The pest protective apparatus 300 of the present invention can also comprise means for distributing water to the at least one fibrous network and/or the at least one fibrous cover 14, the means in various instances comprising a cartridge 16. The cartridge 16 is configured for being in fluid connection with the at least one fibrous network and/or the at least one fibrous cover 14 and for being fed with water. The cartridge 16 can comprise an inlet 18 and an outlet 20.

Depending whether the pest protective apparatus 300 comprises or does not comprise the cartridge 16, the apparatus is configured either to transform at least one precursor 8, which is incorporated to the preparation 100 able to produce biopesticide and/or repellent for controlling plant pathogens by diffusion through the fibre 200, into a biopesticide and/or a repellent product 10; or to incorporate the precursor 8 into the cartridge 16.

Depending whether the pest protective apparatus 300 comprises or does not comprise the cartridge 16, the at least one mineral and/or the at least one cofactor indispensable for the enzyme activity are incorporated either directly within the hydrophilic gel 6 of the biopesticide and/or repellent preparation 100; or within the cartridge 16.

In the case of the preparation 100 able to produce biopesticide and/or repellent for controlling plant pathogens, in the case of the fibres, as well as in the case of the pest protective apparatus of the present invention, the enzymatic reaction is triggered by the addition of water, which plays the role of the solvent of the reaction, bringing subsequently the substrate 8 into contact with the fibres 200 of the preparation 100 able to produce biopesticide and/or repellent for controlling plant pathogens, and more particularly, into contact with the active site of the enzyme 4.

The second utility of water is to bring to the enzyme 4 the at least one mineral and/or the at least one cofactor indispensable for its activity.

Water can come from the rain and/or from artificial means, such for example a drain pump, a hosepipe and/or an irrigation system, and can be channelled to contact the preparation 100 able to produce biopesticide and/or repellent for controlling plant pathogens, triggering subsequently the enzymatic reaction.

When the pest protective apparatus 300 comprises a cartridge 16, the flux of water reaches the cartridge 16 through the inlet 18 which is provided on the cartridge.

Once the enzymatic reaction is over, the product 10, which presents biopesticide and/or repellent properties, is released. Generally, the product is a volatile organic compound.

This product acts to eliminate (in case of biopesticide activity) and/or to keep away (in case of repellent activity) the pests and/or the plant pathogens from the living plants, such as bacteria, fungi, insects, bugs, and/or nematodes.

The biopesticide and/or repellent product 10 resulting from the activation of the precursor by addition of water to the preparation 100 taught in the present invention is applied to plants, in various instances vegetables and/or fruits, in order to prevent pest attacks.

1-15. (canceled)

16. A preparation able to produce at least one insecticides and nematicides for controlling plant pathogens, said preparation comprising:
a) at least one nanoporous material, and
b) at least one active enzyme selected from the group of glycosidase, the at least one active enzyme selected from the group of glycosidase being configured to transform at least one precursor into at least one of insecticides and nematicides product, wherein the at least one active enzyme selected from the group of glycosidase is immobilized within the at least one nanoporous material.

17. The preparation according to claim 16, wherein the at least one nanoporous material is embedded within one hydrophilic gel,

18. The preparation according to claim 17, wherein the hydrophilic gel is made of at least one of agarose, agar-agar, alginate, pectin, starch and gelatine.

19. The preparation according to claim 16, wherein the at least one active enzyme from the group of glycosidase is thioglucosidase.

20. The preparation according to claim 16, wherein the at least one active enzyme, selected from the group of glycosidase, immobilized within the at least one nanoporous material, is one of covalently bounded to the at least one nanoporous material, physically adsorbed to the at least one nanoporous material, encapsulated within the at least one nanoporous material, and entrapped within the at least one nanoporous material.

21. The preparation according to claim 16, wherein the active enzyme, selected from the group of glycosidase, immobilized within the at least one nanoporous material is cross-linked with glutaraldehyde or with 1-ethyl-3-(3-dimethylaminopropyl)carbodiimide and N-hydroxysuccinimide.

22. The preparation according to claim 16, wherein the at least one nanoporous material is a silica-based mesoporous material.

23. The preparation according to claim 16, wherein the preparation one of comprises or is adapted to receive, at least one of the at least one precursor, at least one mineral, and at least one cofactor.

24. The preparation according to claim 23, wherein at least one of the at least one mineral and the at least one cofactor is one of a phosphate buffer and an ascorbic acid.

25. A fibre adapted to control plant pathogens, wherein the fibre is coated with at least one preparation able to produce at least one of insecticides and nematicides for controlling plant pathogens, wherein the preparation comprising
a) at least one nanoporous material, and
b) at least one active enzyme selected from the group of glycosidase, the at least one active enzyme selected from the group of glycosidase being configured to transform at least one precursor into the at least one of insecticides and nematicides product, wherein
the at least one active enzyme selected from the group of glycosidase is immobilized within the at least one nanoporous material.

26. The fibre according to claim 25, wherein the at least one preparation is further coated with a protective layer.

27. The fibre according to claim 26, wherein the protective layer is a hydrophobic layer.

28. A pest protective apparatus, said apparatus comprising at least one of at least one fibrous network and at least one fibrous cover, wherein the at least one of the at least one fibrous network and the at least one fibrous cover is made of fibres adapted to control plant pathogens, wherein the fibre is coated with at least one preparation able to produce at least one of insecticides and nematicides for controlling plant pathogens, wherein the preparation comprising
a) at least one nanoporous material, and
b) at least one active enzyme selected from the group of glycosidase, the at least one active enzyme selected from the group of glycosidase being configured to transform at least one precursor into the at least one of insecticides and nematicides product, wherein the at least one active enzyme selected from the group of glycosidase is immobilized within the at least one nanoporous material.

29. The pest protective apparatus according to claim 28, wherein the pest protective apparatus further comprises a means for distributing water to at least one of the fibrous network and the fibrous cover.

30. The pest protective apparatus according to claim 29, wherein the means comprises a cartridge configured for being in fluid connection with at least one of the at least one fibrous network and the at least one fibrous cover and for being fed with water.

31. A method for controlling plant pathogens with a preparation able to produce at least one of insecticides and nematicides for controlling plant pathogens, wherein said method comprises the following steps:
activating at least one precursor by addition of water to the preparation able to produce at least one of insecticides and nematicides for controlling plant pathogens, wherein the preparation comprises:
a) at least one nanoporous material, and
b) at least one active enzyme selected from the group of glycosidase, the at least one active enzyme selected from the group of glycosidase being configured to transform at least one precursor into the at least one of insecticides and nematicides product, wherein the at least one active enzyme selected from the group of glycosidase is immobilized within the at least one nanoporous material; and
applying the at least one of the insecticides and the nematicides product to plants in order to prevent pest attacks.

32. The method for controlling plant pathogens according to claim 31, wherein the at least one precursor is comprised into the preparation able to produce the at least one of the insecticides and the nematicides for controlling plant pathogens.

33. The method for controlling plant pathogens according to claim 31, wherein the preparation also comprises at least one of at least one mineral and at least one cofactor.

34. The method for controlling plant pathogens according to claim 31, wherein the at least one precursor is incorporated into the cartridge of a pest protective apparatus, wherein the pest protective apparatus comprises at least one of at least one fibrous network and at least one fibrous cover, wherein the at least one of the at least one fibrous network and the at least one fibrous cover is made of fibres adapted to control plant pathogens, wherein the fibre is coated with at least one preparation able to produce the at least one of insecticides and nematicides for controlling plant pathogens, wherein the preparation comprising
a) at least one nanoporous material, and
b) at least one active enzyme selected from the group of glycosidase, the at least one active enzyme selected from the group of glycosidase being configured to transform at least one precursor into the at least one of insecticides and nematicides product, wherein the at least one active enzyme selected from the group of glycosidase is immobilized within the at least one nanoporous material.

35. The method for controlling plant pathogens according to claim 34, wherein the at least one precursor is incorporated into the cartridge of the pest protective apparatus with at least one of at least one mineral and at least one cofactor.
